(19) United States
(12) Patent Application Publication
(10) Pub. No.: US 2019/0249229 A1
(43) Pub. Date: Aug. 15, 2019

(54) BAM SIGNATURES FROM LIQUID AND SOLID TUMORS AND USES THEREFOR

(71) Applicants: Nant Holdings IP, LLC, Culver City, CA (US); Nantomics, LLC, Culver City, CA (US)

(72) Inventors: Patrick Soon-Shiong, Los Angeles, CA (US); Shahrooz Rabizadeh, Los Angeles, CA (US)

(21) Appl. No.: 15/894,171

(22) Filed: Feb. 12, 2018

(51) Int. Cl. C12Q 1/6809; C12Q 1/6869; G16H 50/20; G06F 19/22

(52) U.S. Cl. CPC C12Q 1/6809; C12Q 1/6869; G01N 2570/00; G06F 19/22; G16H 50/20

(57) ABSTRACT

Treatment of a patient diagnosed with cancer is monitored by comparing sequence data from liquid biopsies obtained during and/or after treatment with tumor and patient specific sequence data from a solid tumor obtained prior to treatment.

### FIELD OF THE INVENTION

The field of the invention is monitoring of treatment of various neoplastic diseases, and especially as they relate to monitoring of ongoing treatment using liquid biopsies.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Genetic testing of tumor tissue prior to treatment of a patient diagnosed with cancer has become relatively common and often includes cancer gene panels, exome sequencing, and even whole genome sequencing. Such testing advantageously allows in at least some cases for highly personalized treatment. However, where whole genome or exome sequencing of tumor tissue is performed, the vast amount of data collected will often present a logistic and/or computational challenge (e.g., tumor genome FASTQ sequence file for 30× coverage is approximately 220 GB). Moreover, continued genetic testing of tumor tissue to monitor treatment progress is generally not performed, due to, among other factors, the risk and discomfort of repeated tumor biopsies, and the even larger quantity of sequence data generated for processing.

To circumvent problems associated with repeated tumor biopsies, cell free or circulating DNA has been recently used as a proxy for tumor biopsies and gained attention to monitor or detect tumor growth. For example, DNA from tumor tissue and cell free DNA (cfDNA) from blood was analyzed for hotspot mutations using a reference genome (hg19) and it was shown that at least for some markers cfDNA was suitable (Clin Canc Res. 2016, OF1-9). However, while the specificity was about 95%, the test had a sensitivity of only 55%. In yet other reports, selected mutations were followed in plasma and overall quantities of circulating DNA correlated with overall survival (NEJM 2013, 368: 1199-1209), and in still another study, genome wide aggregated allelic loss and point mutations against a reference genome (hg18) were detected and quantified using shotgun sequencing. Here, fractional concentrations of tumor-derived DNA in plasma were determined, and the so obtained values were correlated with tumor size and surgical treatment (Clin Chem. 2013, 59:1, 211-224). Elsewhere, certain circulating tumor DNA (ctDNA) biomarkers were reported for selected gynecologic cancers (PLoS ONE 10(12): e0145754) to identify tumor status.

To select tumor markers, US2016/0032396 teaches statistical methods to identify cancer associated mutation patterns that can be detected from circulating tumor DNA. In yet another approach, copy number variation analyses were described in US2017/0211153 for prediction of treatment response using urine and plasma samples. While such methods allow for some insight into tumor presence or status, various difficulties nevertheless remain. Among other problems, tumors are often genetically heterogeneous and tend to change and/or undergo clonal selection during treatment, which is typically not readily monitored using conventional methods where cell free DNA is analyzed. Moreover, the use of reference genomes (e.g., hg18 or 19) will further compound issues associated with identifications of mutations that are genuine to the tumor.

Thus, even though numerous methods of genetic testing of cell free DNA are known in the art for patients diagnosed with cancer, various disadvantages nevertheless remain. Therefore, there is still a need for improved systems and methods of cfDNA based testing, and particularly where such testing is employed to monitor ongoing treatment of a patient.

### SUMMARY OF THE INVENTION

The inventive subject matter is directed to methods and systems of monitoring treatment of cancer using sequence information of a solid tumor that is collected prior to treatment, and subsequent sequence information from liquid biopsies during and after treatment, wherein the sequence information of the liquid biopsies is preferably obtained by deep (e.g., at least 50×, or at least 100×) whole exome sequencing, Moreover, it is generally preferred that the sequence information of the liquid biopsies is compared against the tumor and patient specific sequence information of the solid tumor as well as against matched normal sequence information of the same patient to so advantageously allow identification of newly arisen mutations and/or clonal selection or expansion.

In one aspect of the inventive subject matter, the inventors contemplate a method of monitoring treatment of a patient that includes a step of obtaining, prior to a treatment, patient and tumor specific mutation data of a solid tumor of a patient, wherein the mutation data are generated from first sequence data of a solid tumor tissue of the patient and second sequence data of matched normal tissue of the patient. In a further step, and during treatment, third sequence data of a liquid biopsy of the patient are obtained, and in yet another step, the third sequence data and at least one of the mutation data and the first sequence data are used to determine a treatment signature. Most typically, the treatment signature is representative of a response to the treatment.

While not limiting to the inventive subject matter, it is generally preferred that the mutation data are generated by incremental synchronous alignment of the first sequence data with the second sequence data, and that the treatment signature is generated by at least one of incremental synchronous alignment of the first sequence data with the third sequence data and incremental synchronous alignment of the second sequence data with the third sequence data. For example, the mutation data may be in VCF format and the treatment signature may be generated by differential analysis of the mutation data against the third sequence data. Most typically, the first and second sequence data are whole genome sequence data or whole exome sequence data, and the first and second sequence data have a read depth of between 10× and 50×, while the third sequence data have a read depth of between 20× and 500×. Where desired, the mutation data and the treatment signature are in VCF format.

In further contemplated aspects, the first and second sequence data are whole genome sequence data, and the third sequence data are whole exome sequence data. Moreover, it is contemplated that the first and second sequence data have a read depth that is less than a read depth of the third sequence data. Commonly, the liquid biopsy is drawn from whole blood, spinal fluid, ascites fluid, or urine. As will be readily appreciated, the liquid biopsy may be further processed to isolate exosomes, cell free DNA, cell free RNA, or circulating tumor cells, and obtaining the third sequence data from the isolated exosomes, cell free DNA, cell free RNA, or circulating tumor cells.

Additionally, it is contemplated that the treatment signature may be determined by comparing the third sequence data with the mutation data, or that the treatment signature may be determined by comparing the third sequence data with the first and second sequence data. In such case, it is preferred that the first, second, and third sequence data are compared by incremental synchronous alignment. In yet further contemplated aspects, the method may additionally include a step of obtaining, during treatment, fourth sequence data of another liquid biopsy of the patient, and another step of using the fourth sequence data and at least one of the mutation data, the first sequence data, and the third sequence data to calculate a second treatment signature that is representative of a later response to the treatment. Where desired, contemplated methods may also comprise a step of identifying a clonal subpopulation in the mutation data and/or in the treatment signature. Moreover, it is contemplated that the step of calculating the treatment signature may include a step of comparing abundance or allele fraction of corresponding mutations between the first and third sequence data, and/or that the step of calculating the treatment signature may include a step of comparing abundance or allele fraction of corresponding mutations between the first, second, and third sequence data. Additionally, the step of calculating the treatment signature may comprise a step of identifying a new mutation in the third sequence data relative to at least one of the first and second sequence data, and/or a step of obtaining, after treatment, post-treatment sequence data from a liquid biopsy of the patient.

Various objects, features, aspects and advantages of the inventive subject matter will become more apparent from the following detailed description of preferred embodiments.

### DETAILED DESCRIPTION

The inventors have now discovered that cancer treatment can be monitored using omics analysis of sequence information obtained from the tumor and matched normal in combination with sequence information obtained from liquid biopsies. In preferred aspects of the inventive subject matter, tumor mutations or a tumor mutation signature is first collected by incremental synchronous alignment of tumor and matched normal tissue of a patient, typically prior to first treatment. After the treatment has started, additional sequence information is obtained, preferably from deep sequencing of a liquid biopsy, for example, from peripheral blood or other biological fluids. The so obtained sequence information of the liquid biopsy is then compared against the sequence information obtained from the tumor (and optionally also from matched normal, or against a condensed output from tumor versus matched normal such as a VCF file) to so arrive at a first treatment signature representative of a treatment response. Moreover, where immune therapy of the cancer comprises DNA vaccination or a treatment using a recombinant virus (e.g., using a recombinant adenovirus), recombinant DNA from the therapy may be monitored as well using deep sequencing of a liquid biopsy.

Of course, it should be appreciated that liquid biopsies contain nucleic acids from various distinct compartments (e.g., DNA and/or RNA from circulating tumor cells, DNA and/or RNA from exosomes, and cell free DNA and/or RNA). Consequently, analyses contemplated herein may not only provide information on changes in the sequence reads from the liquid biopsies, but also information with respect to the source of the changed sequence reads (e.g., reduction in circulating tumor cells and/or exosomes). Additionally, analyses contemplated herein will also allow for identification of subclonal populations in the tumor and/or liquid biopsies (e.g., via determination of (relative) abundance or allele frequencies), and with that provide information as to the selectivity or selective efficacy of the treatment with respect to the subclonal populations.

Advantageously, the omics data from all sources (i.e., tumor tissue, normal tissue, liquid biopsy) will have a sufficient read depth to so allow for statistically significant determination of allele frequencies and/or ploidy (allele/gene/chromosomal copy numbers). Such determination will advantageously be performed from aligned reads, where such alignment is either against a human reference sequence and/or against matched normal. For example, raw sequence reads can be analyzed against a human reference sequence (e.g., hg18 or hg19) to identify sample versus reference mutations, raw sequence reads can be aligned in a BAM or SAM format for subsequent comparison with another set of sequence reads in BAM or SAM format to so identify a patient and tumor specific mutation in a, for example, incremental synchronous alignment. Thus, omics data most preferably will be in GAR, SAM, or BAM format. With respect to the read depth of the omics data from the liquid biopsy, it is generally contemplated that the read depth is equal or greater than the read depth for the tumor and matched normal tissue of the same patient, and in most instances significantly greater. For example, suitable read depths for the omics data from the liquid biopsy are at least 20×, or at least 50×, or at least 70×, or at least 100×, or at least 150×, or at least 150×, or at least 200×, or at least 250×, or at least 300×, or at least 400×, or at least 500×. Viewed form a different perspective, contemplated read depths will be between 20-50×, or between 50-100×, or between 100-200×, or between 200-500×, or even higher. Therefore, the ratio of the read depth for the tumor/matched normal tissue and the read depth for the liquid biopsy will be at least 1:2, or at least 1:3, or at least 1:5, or at least 1:10, or at least 1:15, or at least 1:20.

In most cases, omics data for the tumor/matched normal tissue will preferably be DNA omics data that may be derived from whole genome sequencing (e.g., pair end sequencing) or whole exome sequencing following standard protocols well known in the art. Alternatively, sequencing may be more limited to selected genes or areas of interest, and suitable selected genes will include known cancer driver genes, inherited cancer risk genes, and genes previously identified in the patient as being mutated regardless of the functional impact of the mutation. Likewise, the omics data for the liquid biopsy will preferably be DNA omics data that may be derived from whole genome sequencing (e.g., pair end sequencing) or whole exome sequencing of DNA obtained from the liquid biopsy (with or without processing to enrich in a specific compartment such as exosomes or circulating cancer cells, or prior amplification step) following standard protocols well known in the art. As before, sequencing of the DNA from the liquid biopsy may also be more limited to selected genes or areas of interest, and suitable selected genes will once more include known cancer driver genes, inherited cancer risk genes, and genes previously identified in the patient as being mutated regardless of the functional impact of the mutation.

Therefore, the omics data for the tumor/matched normal tissue and the liquid biopsy may all be whole genome or whole exome sequence data, or the omics data for the tumor/matched normal tissue and the liquid biopsy may be whole genome or whole exome sequence data while the omics data for the liquid biopsy may be limited to selected genes or areas of interest (e.g., to cancer driver genes, inherited cancer risk genes, genes identified in the tumor/matched normal analysis as being mutated). Additionally or alternatively, it is further contemplated that the omics data for the liquid biopsy may also include transcriptomics data, and especially transcriptomics data covering substantially the entire (i.e., at least 90%, or at least 95%) transcriptome. Such RNA information may advantageously provide in addition to sequence information also data on strength of expression or data on absolute or relative abundance of a gene carrying a mutation identified in the tumor/matched normal analysis. Moreover, use of RNA and transcriptomics in contemplated methods will also allow detection of new and/or recurrent mutations before they become clinically observable using conventional imaging and/or biopsy procedures.

More specifically, and with respect to the cell free DNA and/or RNA it is contemplated that tumor cells and/or some immune cells interacting or surrounding the tumor cells release cell free DNA/RNA to a patient's bodily fluid, and thus may increase the quantity of the specific cell free DNA/RNA in the patient's bodily fluid as compared to a healthy individual. As used herein, the patient's bodily fluid includes blood, serum, plasma, mucus, cerebrospinal fluid, ascites fluid, saliva, and urine of the patient. Alternatively, it should be noted that various other bodily fluids are also deemed appropriate so long as cell free DNA/RNA is present in such fluids. Moreover, the patient's bodily fluid may be fresh or preserved/frozen.

The cell free DNA/RNA typically comprises whole genome, whole exome, and/or whole transcriptome nucleic acids and may therefore may include any types of DNA/RNA that are circulating in the bodily fluid of a person without being enclosed in a cell body or a nucleus. Most typically, the source of the cell free DNA/RNA is the tumor cells. However, it is also contemplated that the source of the cell free DNA/RNA is an immune cell (e.g., NK cells, T cells, macrophages, etc.). Thus, the cell free DNA/RNA can be circulating tumor DNA/RNA (ctDNA/RNA) and/or circulating free DNA/RNA (cf DNA/RNA, circulating nucleic acids that do not derive from a tumor). While not wishing to be bound by a particular theory, it is thought that release of cell free DNA/RNA originating from a tumor cell may be increased when the tumor cell interacts with an immune cell or when the tumor cells undergo cell death (e.g., necrosis, apoptosis, autophagy, etc.). Thus, in some embodiments, the cell free DNA/RNA may be enclosed in a vesicular structure (e.g., via exosomal release of cytoplasmic substances) so that it can be protected from nuclease (e.g., RNAase) activity in some type of bodily fluid. Yet, it is also contemplated that in other aspects, the cell free DNA/RNA is a naked DNA/RNA without being enclosed in any membranous structure, but may be in a stable form by itself or be stabilized via interaction with one or more non-nucleotide molecules (e.g., any RNA binding proteins, etc.).

Cell free DNA may include any whole or fragmented genomic DNA, or mitochondrial DNA, and cell free RNA may include mRNA, tRNA, microRNA, small interfering RNA, long non-coding RNA (lncRNA). Most typically, the cell free DNA is a fragmented DNA typically with a length of at least 50 base pair (bp), 100 base pair (bp), 200 bp, 500 bp, or 1 kbp. Also, it is contemplated that the cell free RNA is a full length or a fragment of mRNA (e.g., at least 70% of full-length, at least 50% of full length, at least 30% of full length, etc.). As noted earlier, cell free DNA/RNA may include any type of DNA/RNA encoding any cellular, extracellular proteins or non-protein elements. However, in at least some aspects, analysis of the DNA and/or RNA may be limited or focused on one or more cancer-related proteins, or inflammation-related proteins. For example, the cell free DNA/mRNA may be full-length or fragments of (or derived from the) cancer associated genes, or genes encoding a full length or a fragment of inflammation-related proteins, or genes encoding DNA repair-related proteins or RNA repair-related proteins, or genes carrying a mutation (e.g., which may result in an encoded neoepitope). Of course, it should be appreciated that the above genes may be wild type or mutated versions, including missense or nonsense mutations, insertions, deletions, fusions, and/or translocations, all of which may or may not cause formation of full-length mRNA when transcribed.

Any suitable methods to isolate and amplify cell free DNA/RNA are contemplated. Most typically, cell free DNA/RNA is isolated from a bodily fluid (e.g., whole blood) that is processed under a suitable conditions, including a condition that stabilizes cell free RNA. Preferably, both cell free DNA and RNA are isolated simultaneously from the same badge of the patient's bodily fluid. Yet, it is also contemplated that the bodily fluid sample can be divided into two or more smaller samples from which DNA or RNA can be isolated separately. Once separated from the non-nucleic acid components, cell free RNA are then quantified, preferably using real time, quantitative PCR or real time, quantitative RT-PCR.

The liquid biopsy typically uses a bodily fluid of the patient, and it should be appreciated that any such fluid can be obtained at any desired time point(s) depending on the purpose of the omics analysis. For example, the bodily fluid of the patient can be obtained before and/or after the patient is confirmed to have a tumor and/or periodically thereafter (e.g., every week, every month, etc.) in order to associate the cell free DNA/RNA data with the prognosis of the cancer. In some embodiments, the bodily fluid of the patient can be obtained from a patient before and after the cancer treatment (e.g., chemotherapy, radiotherapy, drug treatment, cancer immunotherapy, etc.). While it may vary depending on the type of treatments and/or the type of cancer, the bodily fluid of the patient can be obtained at least 24 hours, at least 3 days, at least 7 days after the cancer treatment. For more accurate comparison, the bodily fluid from the patient before the cancer treatment can be obtained less than 1 hour, less than 6 hours before, less than 24 hours before, less than a week before the beginning of the cancer treatment. In addition, a plurality of samples of the bodily fluid of the patient can be obtained during a period before and/or after the cancer treatment (e.g., once a day after 24 hours for 7 days, etc.).

With respect to sequence analysis of the omics data from the tumor tissue, the matched normal tissue (e.g., corresponding non-cancerous tissue or blood from the same patient), and the liquid biopsy, it should be appreciated that all manners of sequence comparison are deemed suitable for use herein and include sequence comparison against an external reference sequence (e.g., hg18, or hg19), sequence comparison against an internal reference sequence (e.g., matched normal), and sequence processing against known common mutational patterns (e.g., SNVs). Therefore, contemplated methods and programs to detect mutations between tumor and matched normal, tumor and liquid biopsy, and matched normal and liquid biopsy include iCallSV (URL: github.com/rhshah/iCallSV), VarScan (URL: varscan.sourceforge.net), MuTect (URL: github.com/broadinstitute/mutect), Strelka (URL: github.com/Illumina/strelka), Somatic Sniper (URL: gmt.genome.wustl.edu/somatic-sniper/), and BAMBAM (US 2012/0059670).

However, in especially preferred aspects of the inventive subject matter, the sequence analysis is performed by incremental synchronous alignment of the first sequence data (tumor sample) with the second sequence data (matched normal), for example, using an algorithm as for example, described in Cancer Res 2013 Oct. 1; 73(19):6036-45, US 2012/0059670 and US 2012/0066001 to so generate the patient and tumor specific mutation data. As will be readily appreciated, the sequence analysis may also be performed in such methods comparing omics data from the liquid biopsy against tumor omics data and/or matched normal omics data to so arrive at an analysis that can not only inform a user of mutations that are genuine to the tumor within a patient, but also of mutations that have newly arisen during treatment (e.g., via comparison of matched normal/liquid biopsy and matched normal/tumor, or via comparison of tumor and liquid biopsy). In addition, using such algorithms (and especially BAMBAM), allele frequencies and/or clonal populations for specific mutations can be readily determined, which may advantageously provide an indication of treatment success with respect to a specific tumor cell fraction or population.

More specifically, in previously known mutation analyses for distinction of a variant as being somatic (i.e., a variant sequence found only in the tumor) or germline (i.e., a variant sequence that is inherited or heritable), massive quantities of data representing reconstructed tumor and matched normal (or other reference) genomes had to be compared. Such task is typically performed sequentially, by alignment and summarizing data at every genomic position for both tumor and germline and then combining the results for analysis. Unfortunately, because whole-genome BAM files are hundreds of gigabytes in their compressed form (1-2 terabytes uncompressed), the intermediate results that would need to be stored for analysis is extremely large and slow to merge and analyze.

In contrast, incremental synchronous alignment methods (e.g., BAMBAM) can read from two, three, or more files (e.g., tumor omics BAM file, matched normal omics BAM file, liquid biopsy omics BAM file) at the same time, constantly keeping each BAM file in synchrony with the other(s) and piling up the genomic reads that overlap every common genomic location between the two files. For each pair of pileups, statistical analyses can be performed to maximize the joint probability of the matched normal genotype (given the germline reads and the reference nucleotide), the tumor genotype (given the germline genotype, a simple mutation model, an estimate of the fraction of contaminating normal tissue in the tumor sample, and the tumor sequence data), and/or the liquid biopsy genotype (given the germline genotype, a simple mutation model, an estimate of the fraction of contaminating normal tissue in the tumor sample, and the tumor and/or normal sequence data).

By processing these massive BAM files with this method, the computer's RAM usage is minimal and processing speed is limited primarily by the speed that the filesystem can read the files available for analysis. This enables processing of massive amounts of data quickly, while being flexible enough to run on a single computer or across a computer cluster. Moreover, it should be appreciated that the analytic output is fairly minimal, preferably comprising only the differences found in each of the files (e.g., in form of a variant call format (VCF) file). Such representation is further beneficial as a whole-genome difference is notated that requires significantly less data storage than it would take if all genome information was stored for each file separately. Indeed, it should be appreciated that the so obtained mutation data in VCF format represent only a very small fraction of whole genome data, however that small fraction of data is highly relevant to the patients tumor.

Even further, it should be noted that the incremental synchronous alignment methods will not require a reconstruction of the respective sequence reads into a full genome, but can be performed from the reads stored in the BAM or SAM file format. Therefore, such contemplated methods are computationally efficient and allow for rapid comparison of three, four, and even more data sets of the same patient without genome reconstruction, even where the read depth is very high (e.g., >50×).

In further contemplated methods, the liquid biopsy omics data need not be subjected to whole genome or exome sequencing, but may be employed to track presence and/or quantity of the patient and tumor-specific mutations using methods specific to the particular mutation. For example, it is contemplated that the specific mutations may be detected using quantitative rtPCT of mutated sequences to quantify the mutations, or allele specific hybridization or allele specific amplification or single nucleotide primer extension to detect presence of the specific mutations (e.g., mutations detected by tumor/matched normal sequencing) from the liquid biopsy sample.

For example, a solid tumor biopsy sample from a patient diagnosed with breast cancer is subjected to whole genome sequencing at a depth of 25× using whole genome sequencing of matched normal tissue (e.g., PMBC from same patient) as a control to so obtain the patient and tumor specific mutation data. Most typically the mutation data are generated by incremental synchronous alignment of the first sequence data (tumor sample) with the second sequence data (matched normal), for example, using BAMBAM as an incremental synchronized alignment algorithm. It should be appreciated that the so obtained mutation data may also be employed in further analysis, and especially pathway activity analysis, to develop a treatment regimen for the patient based on the information obtained from the mutation data. For example, preferred pathway activity data analysis can be done using PARADIGM as described in Bioinformatics 2010 Jun. 15; 26(12): i237-i245, Bioinformatics 2013 Jul. 1; 29(13): i62-i70, and WO 2013/062505. Thus, a treatment regime is established for the patient using mutation information and/or pathway activity analysis, along with further suitable methods, including transcriptomics or transcriptome analysis (e.g., using RNAseq), proteomics analysis (using selected reaction monitoring or other mass spectroscopic method), immunohistochemical analysis (e.g., FISH, ELISA) and/or selected enzymatic activity assays (e.g., to determine kinase or phosphatase activity.

After initiation of treatment, it is then contemplated that one or more liquid biopsies are taken from the patient and that the so obtained biopsies are subjected to further genetic analysis. For example, suitable liquid biopsy samples include various biological fluids, and especially whole blood, a white blood cell fraction of whole blood, spinal fluid, ascites fluid, and urine. All of such biological fluids are known to include various nucleic acids, and it is expected that at least a small fraction of the nucleic acids will be derived from the solid tumor, for example, in form of circulating tumor cells, exosomes, microvesicles, and/or cell free (typically lipoprotein-associated) DNA. It should be noted that source of the nucleic acids may be informative of the status of the solid tumor (or metastasis from the tumor). For example, distressed tumor cells are known to shed exosomes and microvesicles, while apoptotic cells are known to produce cell free DNA. Likewise, tumors may (in progression to establishing metastases) release circulating tumor cells. Thus, it should be noted that the liquid biopsy material may be further processed to isolate or enrich exosomes, cell free DNA, or circulating tumor cells, from which then the third sequence data may be obtained. Of course, such processing need not be performed where not desired.

With respect to the step of obtaining third sequence from the liquid biopsy sample, it is contemplated that the sequence data are generated from whole genome sequencing, from whole exome sequencing, and/or from transcriptome sequencing as noted above. As the tumor related fraction of nucleic acids in the liquid biopsy is expected to be relatively low, it is typically preferred that the sequencing of the nucleic acids in the liquid biopsy is performed to a depth that is greater that the sequencing depth of the solid tumor (for generation of the mutation data) as already discussed above. For example, suitable sequencing depths for the first and second sequence data will typically be between 1× and 100×, and more typically between 10× and 70×, and most typically between 20× and 50×. Thus, suitable sequencing depths for the first and second sequence data will be equal or less than 70×, more typically equal or less than 50×, and most typically equal or less than 30×. Conversely, it is preferred that the sequencing depth for generation of the third sequence data will be at least 20×, more typically at least 50×, even more typically at least 100×, and most typically at least 150×. For example, contemplated sequencing depths for generation of the third sequence data will be between 25×-50×, or between 50×-100×, or between 100× and 300×, and even higher.

Moreover, and as also noted above, while whole genome or whole exome sequencing is generally preferred, it should be appreciated that targeted sequencing that only covers the mutations identified in the mutation data is also contemplated herein. Thus, it should be recognized that in contemplated systems and methods tumor data (from the mutation data) are employed as reference against subsequent sequence data from liquid biopsies. Such analysis dramatically reduces compute time and storage requirements of nucleic acid data, and allows for substantially simplified downstream analysis.

For example, the first and second sequence data may be whole genome sequence data, while the third sequence data may be whole exome sequence data. In such systems, the third sequence data may be compared with the mutation data to so obtain a treatment signature. Alternatively, the treatment signature may also be calculated by comparing the third sequence data with first and second sequence data, preferably using incremental synchronous alignment as discussed above. Regardless of the particular manner of comparison, it should be recognized that in addition to the third sequence data, further fourth, fifth, sixth, etc. sequence data may be obtained from one or more subsequent liquid biopsies. Therefore, liquid biopsies may be performed in any time interval during, and even post treatment to so produce multiple treatment signatures, which may be employed to generate, modify or update a treatment regimen. These treatment signatures can also be analyzed for the response of the cancer to the treatment and/or to identify trends in the circulating tumor cells, cell free DNA, and/or exosomes, which may be informative about the source and state of the tumor cells from which these entities are derived.

Moreover, it should be recognized that the mutation data may also inform a practitioner about the presence and/or quantity of clonal subpopulations within the solid tumor. As it is unfortunately expected that not all cells of all subpopulations in the solid tumor will be equally responsive to the treatment, increase and/or decrease of subpopulations during treatment can be readily monitored using contemplated systems and methods. For example, using the incremental synchronous alignment methods, information on allele frequencies and/or abundance of specific mutations can be detected, which will correlate with number of tumor cells or tumor size and with clonal fractions characterized by specific mutations. Moreover, such methods will also allow tracing of new mutations, either arising from a tumor cell population or de novo as a new tumor clone. Thus, emergence of new subpopulations and emerging metastases can be followed by quantitative and/or qualitative analysis of the third and subsequent sequence data in comparison with the mutation data and/or first and/or second sequence data. In many cases, the omics data of the liquid biopsies will be a quantifiable indicator well before new tumor cones or metastases can be clinically detected (e.g., by imaging methods or biopsy/surgery). Treatment can then be adjusted or updated in response to the newly determined treatment signature. Lastly, it is contemplated that the third and subsequent sequence data may be obtained, for example, to ascertain or confirm progression free survival.

In general, and with respect to the file format of the sequence data, it is preferred that the format is a BAM, SAM, or FASTA format. Regardless of the nature of the particular sequence format, it is generally contemplated that all nucleic acid sequences referred herein are stored on a database for retrieval by an analysis engine, and such database may be a single or a distributed database. Thus, the term ‘database’ should be understood as not being limited to a single physical device, but to include multiple and distinct storage devices that are informationally coupled to each other. It should further be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

Consequently, the inventors also contemplate a method in which an analysis engine is informationally coupled to a sequence database that stores first, second, and/or third sequence data. The analysis engine is then programmed to generate mutation data of a solid tumor of a patient from first and second sequence data, wherein the first sequence data are from a solid tumor tissue of the patient and the second sequence data are from a matched normal tissue of the patient. The analysis engine is further programmed to calculate a treatment signature that is representative of a response to the treatment, wherein the treatment signature is calculated from a comparison between third sequence data of a liquid biopsy and at least one of the mutation data and the first sequence data. Of course, in such systems and methods as discussed above, it should be appreciated that the mutation data of the solid tumor of the patient from the first and second sequence data are not necessarily required, but that the first, second, and third sequence data may be analyzed together in one step of such methods.

It should be recognized that contemplated systems and methods, particularly when used in conjunction with incremental synchronous alignment as described above, substantially increase processing speed in a computational system used for such analysis. It should be noted that the complexity of the analysis and the enormous size of sequence data files will render such method entirely unsuitable for human practice as such file analysis only would readily exceed the lifespan of a human, even if one would analyze 10,000s of bases per day. Moreover, further comparison with additional sequence data, even though possibly much smaller, would further add to the impossibility of human action. In addition, it should be pointed out that the use of mutation data as reference for subsequent third and further sequence data from liquid biopsies will have the technical effect of drastically improving analysis time as such files (a) can be rapidly processed without much memory demand as compared to loading an entire sequence into memory, (b) allows for rapid analysis of genomic changes over time without causing patient discomfort due to multiple biopsies, and (c) will allow for identification of new mutations, of mutation abundance, and of allele fractions. Additionally, contemplated systems and methods allow for the first time a real time and dynamic analysis of treatment response as observed through the nucleic acid content in the liquid biopsies. Lastly, it is noted that upon identification of further changes in sequence data of the liquid biopsy, the so obtained result may be used to model in silico a potential impact of a new treatment regimen.

Therefore, it should be appreciated that a treatment signature that is representative of a response to the treatment can be established by comparison of the various omics data from one or more liquid biopsies against the mutation data (that are typically generated by comparison of tumor versus matched normal), and or by comparison against the matched normal omics data and/or against the tumor data. Viewed from a different perspective, the treatment signature may reflect presence, absence, increase, and/or decrease of specific mutations in the liquid biopsy data as compared to the mutation data. Such indication advantageously allows for tracking of treatment efforts with respect to one or more specific mutations (and with that possibly also with respect to one or more subclones in the tumor). Additionally, the treatment signature may also indicate new mutations that have arisen from normal cells (e.g., new mutation in liquid biopsy omics data relative to matched normal omics data) and/or new mutations that have arisen from tumor cells (e.g., new mutation in liquid biopsy omics data relative to tumor omics data). Likewise, where the analysis is based on omics data from tumor, matched normal, and liquid biopsy or biopsies, a treatment signature may also provide a dynamic analysis with respect to presence and absence of mutations during or after treatment, and their allele fractions.

### Example

DNA isolation from tumor and matched normal: A fresh tumor tissue sample is obtained via surgical procedure, either during resection or by biopsy following routine clinical protocol. Using the so obtained tissue specimen genomic DNA is isolated following the instructions of a commercially available DNA isolation kit (e.g., QIAGEN DNeasy Blood & Tissue Kit).

DNA/RNA isolation from liquid biopsy: 10 ml of whole blood is drawn into a test tube, and cell free DNA and RNA is isolated following the instructions of a commercially available DNA isolation kit (STRECK CELL-FREE DNA BCT and CELL-FREE RNA BCT). Cell free RNA is stable in whole blood in the cell-free RNA BCT tubes for seven days while cell free RNA is stable in whole blood in the cell-free DNA BCT tubes for fourteen days, allowing time for shipping of patient samples from world-wide locations without the degradation of cell free RNA.

Moreover, it is generally preferred that the cell free RNA is isolated using RNA stabilization agents that will not or substantially not (e.g., equal or less than 1%, or equal or less than 0.1%, or equal or less than 0.01%, or equal or less than 0.001%) lyse blood cells. Viewed from a different perspective, the RNA stabilization reagents will not lead to a substantial increase (e.g., increase in total RNA no more than 10%, or no more than 5%, or no more than 2%, or no more than 1%) in RNA quantities in serum or plasma after the reagents are combined with blood. Likewise, these reagents will also preserve physical integrity of the cells in the blood to reduce or even eliminate release of cellular RNA found in blood cell. Such preservation may be in form of collected blood that may or may not have been separated. In less preferred aspects, contemplated reagents will stabilize cell free RNA in a collected tissue other than blood for at 2 days, more preferably at least 5 days, and most preferably at least 7 days. Of course, it should be recognized that numerous other collection modalities are also deemed appropriate, and that the cell free RNA can be at least partially purified or adsorbed to a solid phase to so increase stability prior to further processing.

The whole blood in 10 mL tubes is centrifuged to fractionate plasma at 1600 rcf for 20 minutes. The so obtained plasma is then separated and centrifuged at 16,000 rcf for 10 minutes to remove cell debris. Of course, various alternative centrifugal protocols are also deemed suitable so long as the centrifugation will not lead to substantial cell lysis (e.g., lysis of no more than 1%, or no more than 0.1%, or no more than 0.01%, or no more than 0.001% of all cells). Cell free RNA is extracted from 2 mL of plasma using Qiagen reagents. The extraction protocol was designed to remove potential contaminating blood cells, other impurities, and maintain stability of the nucleic acids during the extraction. All nucleic acids were kept in bar-coded matrix storage tubes, with DNA stored at −4° C. and RNA stored at −80° C. or reverse-transcribed to cDNA that is then stored at −4° C. Notably, so isolated cell free RNA can be frozen prior to further processing.

Sequencing: DNA samples for tumor and matched normal are subjected to whole genome sequencing using standard protocols for next generation sequencing on an Illumina NovaSeq 6000 System sequencer. Likewise, where RNA sequences are obtained from the liquid biopsy, RNA-seq is performed using standard protocols for next generation sequencing on an Illumina HiSeq 4000 System. The raw data (e.g., BCL or FASTQ format) are converted using SAMtools to respective BAM files for further analysis.

RNA analysis of specific mutated genes: With respect to the transcription strength (expression level), transcription strength of the cell free RNA can be examined by quantifying the cell free RNA. Quantification of cell free RNA can be performed in numerous manners, however, expression of analytes is preferably measured by quantitative real-time RT-PCR of cell free RNA using primers specific for each gene. For example, amplification can be performed using an assay in a 10 μL reaction mix containing 2 μL cell free RNA, primers, and probe. mRNA of α-actin can be used as an internal control for the input level of cell free RNA. A standard curve of samples with known concentrations of each analyte was included in each PCR plate as well as positive and negative controls for each gene. Test samples were identified by scanning the 2D barcode on the matrix tubes containing the nucleic acids. Delta Ct (dCT) was calculated from the Ct value derived from quantitative PCR (qPCR) amplification for each analyte subtracted by the Ct value of actin for each individual patient's blood sample. Relative expression of patient specimens is calculated using a standard curve of delta Cts of serial dilutions of Universal Human Reference RNA set at a gene expression value of 10 (when the delta CTs were plotted against the log concentration of each analyte). Alternatively, as described above RNA analysis can be performed using RNA-seq.

Omics Analysis: BAM files are processed using Contraster (NantOmics, LLC, Santa Cruz, Calif., USA) to identify mutations and abundance/allele frequencies for mutations between tumor and matched normal (to identify patient and tumor specific mutations), for mutations between liquid biopsy and matched normal (to identify newly arisen mutations vis-à-vis normal), between liquid biopsy and tumor (to identify newly arisen mutations vis-à-vis tumor), and between matched normal, tumor, and liquid biopsy (to identify and quantify all mutations over time and tissue).

As will be readily apparent, and based on the comparisons, the treatment signature may indicate that specific tumor cells were successfully eradicated with the treatment, or that specific tumor cells remained resistant to treatment, and/or that new mutations arose from an existing tumor and/or from health cells. Accordingly, patient treatment can be adjusted.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of monitoring treatment of a patient, comprising:
obtaining, prior to a treatment, patient and tumor specific mutation data of a solid tumor of a patient;
wherein the mutation data are generated from first sequence data of a solid tumor tissue of the patient and second sequence data of matched normal tissue of the patient;
obtaining, during treatment, third sequence data of a liquid biopsy of the patient; and
using the third sequence data and at least one of the mutation data, the first sequence data, and the second sequence data to determine a treatment signature that is representative of a response to the treatment.

2. The method of claim 1 wherein the mutation data are generated by incremental synchronous alignment of the first sequence data with the second sequence data, and wherein the treatment signature is generated by at least one of incremental synchronous alignment of the first sequence data with the third sequence data and incremental synchronous alignment of the second sequence data with the third sequence data.

3. The method of claim 1 wherein the mutation data are in VCF format and wherein the treatment signature is generated by differential analysis of the mutation data against the third sequence data.

4. The method of claim 1 wherein the first and second sequence data are whole genome sequence data or whole exome sequence data, and wherein the first and second sequence data have a read depth of between 10× and 50×.

5. The method of claim 1 wherein the third sequence data have a read depth of between 20× and 500×.

6. The method of claim 1 wherein the mutation data and the treatment signature are in VCF format.

7. The method of claim 1 wherein the first and second sequence data are whole genome sequence data, and wherein the third sequence data are whole exome sequence data.

8. The method of claim 1 wherein the first and second sequence data have a read depth that is less than a read depth of the third sequence data.

9. The method of claim 1 wherein the liquid biopsy is drawn from whole blood, spinal fluid, ascites fluid, or urine.

10. The method of claim 1 wherein the treatment signature is determined by comparing the third sequence data with the mutation data.

11. The method of claim 1 wherein the treatment signature is determined by comparing the third sequence data with the first and second sequence data.

12. The method of claim 11 wherein the first, second, and third sequence data are compared by incremental synchronous alignment.

13. The method of claim 1 further comprising a step of obtaining, during treatment, fourth sequence data of another liquid biopsy of the patient, and using the fourth sequence data and at least one of the mutation data, the first sequence data, and the third sequence data to calculate a second treatment signature that is representative of a later response to the treatment.

14. The method of claim 1 further comprising a step of identifying a clonal subpopulation in the mutation data or in the treatment signature.

15. The method of claim 14 further comprising a step of using the third sequence data to calculate a treatment signature that is representative of a response of the clonal subpopulation to the treatment.

16. The method of claim 1 further comprising a step of processing the liquid biopsy to isolate exosomes, cell free DNA, cell free RNA, or circulating tumor cells, and obtaining the third sequence data from the isolated exosomes, cell free DNA, cell free RNA, or circulating tumor cells.

17. The method of claim 1 wherein the step of calculating the treatment signature comprises comparing abundance or allele fraction of corresponding mutations between the first and third sequence data.

18. The method of claim 1 wherein the step of calculating the treatment signature comprises comparing abundance or allele fraction of corresponding mutations between the first, second, and third sequence data.

19. The method of claim 1 wherein the step of calculating the treatment signature comprises identifying a new mutation in the third sequence data relative to at least one of the first and second sequence data.

20. The method of claim 1 further comprising a step of obtaining, after treatment, post-treatment sequence data from a liquid biopsy of the patient.
